(19) United States
(12) Patent Application Publication
(10) Pub. No.: US 2014/0017767 A1
(43) Pub. Date: Jan. 16, 2014

(54) INOCULUM AND METHOD OF PREPARATION

(71) Applicant: AMS Laboratories Pty Ltd, Silverwater (AU)

(72) Inventors: Paul PRISCOTT, Silverwater (AU); Yi-hong WANG, Silverwater (AU)

(73) Assignee: AMS Laboratories Pty Ltd, Silverwater (AU)

(21) Appl. No.: 13/924,268

(22) Filed: Jun. 21, 2013

Related U.S. Application Data
(63) Continuation of application No. PCT/AU2011/001629, filed on Dec. 19, 2011.

(30) Foreign Application Priority Data
Dec. 23, 2010 (AU) 2010905639

(51) Int. Cl. C12N 1/20; C12N 1/16; C12N 1/18; C12N 1/14

(52) U.S. Cl. CPC C12N 1/20; C12N 1/14; C12N 1/16; C12N 1/18
USPC 435/252.5; 435/252.1; 435/252.7; 435/252.8; 435/252.9; 435/253.3; 435/253.4; 435/256.1; 435/255.4; 435/255.2

(57) ABSTRACT

The present invention relates to microbial inocula and in particular to standardised inocula for producing reference cultures of microorganisms. The invention has been developed primarily for use as a solid, disc-shaped, inoculum and will be described hereinafter with reference to this application. However, it will be appreciated that the invention is not limited to this particular field of use.

This application is a continuation of PCT/AU2011/001629, filed Dec. 19, 2011; which claims the priority of Australian Application No. 2010905639, filed Dec. 23, 2010. The contents of the above-identified applications are incorporated herein by reference in their entireties.

### FIELD OF THE INVENTION

The present invention relates to microbial inocula and in particular to standardised inocula for producing reference cultures of microorganisms.

The invention has been developed primarily for use as a solid, disc-shaped, inoculum and will be described hereinafter with reference to this application. However, it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

The maintenance of live microorganisms in a stable form is critical for microbiological studies. The methods for the freeze-drying of microorganisms have been relatively successful. Because successfully freeze-dried cells can be stored in the absence of freezing conditions, these procedures are more convenient and less expensive in comparison to super-cooled systems such as liquid nitrogen storage systems.

Generally, cryoprotectants are included in the freezing medium when cells are frozen in order to prevent the damaging effects of water crystals. It is generally believed that the major trauma in preparing a stable freeze dried inoculum of cells occurs as the result of the physical impact on the cells during the freezing and thawing processes. Alteration of the constituents of the cryoprotectant may improve cell viability so that the freezing medium becomes a cryopreservation medium useful for maintaining a more stable quantitative inoculum.

Microbiology laboratories routinely use standardized reference cultures providing a predetermined quantity of microorganisms in quality control processes, for example in processes demonstrating the efficacy of testing methods and culture media. These reference cultures are generally prepared by diluting a culture of microorganisms to obtain a fresh cell suspension that contains an estimated number of colony-forming units per milliliter (CFU/ml). The accuracy with which the number of colony forming units per milliliter can be determined often varies greatly due to the extrapolation of small measurement errors during dilution but also due to the biological variability of the sample per se. As such, using subsequently prepared, fresh reference cultures naturally increases the potential for false or invalid results as it is difficult to consistently determine the number of colony forming units per milliliter for each single inoculum in a series of experiments.

Known inocula to produce reference cultures for the microbiology industry include products such as Bioball® (BTF). Bioball® can provide a plurality of inocula containing a relatively precise and consistent number of microorganisms with a reproducible amount of variation (<2 standard deviations from the mean). However, the production processes for Bioball® are complex and the product precision requirements during preparation make the product relatively expensive to produce.

It is an object of the present invention to overcome or ameliorate at least one of the disadvantages of the prior art, or to provide a useful alternative.

### SUMMARY OF THE INVENTION

The present invention in various embodiments relates to a solid, disc-shaped, microbial inoculum, to improved cryoprotectant formulations of cryopreservation media and to processes for reliably and easily producing such an inoculum. The inoculum is relatively inexpensive to produce in a more user-friendly form, shaped and sized to enable a user to conveniently and accurately transfer a predetermined amount of inoculating material, such as a predetermined number of cells or microorganisms, from one container to another, using standard laboratory equipment.

In a first aspect the present invention provides a solid, dry inoculum comprising a cryopreservation medium and a predetermined amount of inoculating material, wherein said inoculum is substantially disc-shaped.

According to a preferred embodiment of the invention, the substantially disc-shaped inoculum is preferably semispherical and includes a convex surface opposed by a corresponding concave surface, thus facilitating easy transfer from one container to another using a standard laboratory inoculating loop.

Conveniently, the inoculum is produced by freeze drying.

The cryopreservation medium typically comprises Bovine Serum Albumin, myo-inositol, beef extract, bacteriological peptone, gelatin, activated charcoal and water. In particular, the preferred formulation of the cryopreservation medium comprises Bovine Serum Albumin and gelatin at about equal parts (i.e. at a ratio of about 1:1). The inoculating material is typically a cell which may be a microorganism such as a bacterium or a fungus and each inoculum contains from about 0 to about 10⁸ cells. Bacterial cells may be selected, without limitation, from the group consisting of Bacillus cereus; B. pumilus; B. subtilis; Bacteroides fragilis; Brevundimonas diminuta; Bordetella bronchiseptica; Burkholderia cepacia; Clostridium perfringens; C. sporogenes; Enterococcus faecalis; E. hirae; Escherichia coli; Geobacillus stearothermophilus; Klebsiella pneumonia; Kocuria rhizophila; Lactobacillus fermentum; Listeria monocytogenes; Micrococcus luteus; Pseudomonas aeruginosa; Salmonella enterica subsp.; Shigella flexneri; Staphylococcus aureus subsp.; and Streptococcus pyogenes. In the case where the cell is a fungal cell, the fungus may be selected, without limitation, from the group consisting of the following strains Aspergillus niger; Candida albicans; and Saccharomyces cerevisiae.

In a second aspect the present invention provides a method of preparing a solid, dry inoculum comprising the steps of:
a) depositing a plurality of aliquots of liquid cryopreservation medium containing a predetermined amount of inoculating material on a surface;
b) drying each of said aliquots deposited said surface to obtain a plurality of solid, dry inocula; and
c) removing said inocula from said surface,
   wherein each said inoculum is substantially disc-shaped.

Preferably the inocula are semispherical in shape following the drying step. The drying is preferably achieved by freeze-drying the deposited aliquots. To produce the desired inocula, the aliquots generally have a volume ranging from about 10 μl to about 100 μl, preferably from about 15 μl to about 60 μl, and contain from about 0 to about 10⁸ cells of a microorganism as inoculating material.

The surface is typically a plastic surface, preferably the polystyrene surface of a standard laboratory polystyrene dish or tray such as a Petri dish or similar.

In a third aspect, the present invention relates to a solid, dry inoculum when prepared by the method of the second aspect.

In a fourth aspect, the present invention provides a container/pack containing a plurality of solid, dry inocula according to the first or third aspect.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

In the context of this specification the term “inoculum” (plural “inocula”) refers to a physical body resulting from drying an aliquot of cryopreservation medium comprising a predetermined amount of inoculating material. The inocula according to one or more preferred embodiments of the invention are defined by their semi-spherical, substantially disc shaped body which allows convenient handling using standard laboratory equipment and are used to inoculate a culture medium.

A “dry” inoculum according to one or more preferred embodiments of the invention has had substantially all liquid removed. Alternatively, the term “dry” when referring to an inoculum according to one or more preferred embodiments of the invention means that the inoculum may contain residual moisture but is sufficiently “dry” such that it can conveniently be handled using standard laboratory equipment.

A “solid” inoculum according to one or more preferred embodiments of the invention is neither liquid nor gaseous and does not flow under its own weight. Alternatively, the term “solid” when referring to an inoculum according to one or more preferred embodiments of the invention means that the inoculum is sufficiently firm such that it maintains its disc shaped body and can conveniently be handled using standard laboratory equipment.

The term “cryopreservation medium” in the context of the present invention refers to a liquid composition capable of protecting and/or minimizing damage of inoculating material contained within the medium from the physical impact during freezing and thawing.

### BRIEF DESCRIPTION OF THE DRAWINGS

A preferred embodiment of the invention will now be described, by way of example only, with reference to the accompanying drawings in which:

FIG. 1 is a top view of a plurality of the substantially semi-spherical, disc-shaped inocula comprising a convex surface opposed by a corresponding concave surface according to a preferred embodiment of the invention;

FIG. 2A is a photograph showing a side view of the substantially semi-spherical, disc-shaped inocula according to a preferred embodiment of the invention in relation to a centimeter/millimeter scale, the inocula comprise a convex surface opposed by a corresponding concave surface;

FIG. 2B is a top view of the inocula shown FIG. 2A;

FIG. 3A is a photograph showing a top view of an inoculum according to a preferred embodiment of the invention showing the inoculum while being transferred in a plastic bacteriological loop with its concave surface facing upwards;

FIG. 3B is a top view similar to that of FIG. 3A showing an empty bacteriological loop next to the loop shown in FIG. 3A; and

FIG. 4 is a photograph showing a perspective view of a number of inocula according to a preferred embodiment of the invention with their concave surfaces facing upwards.

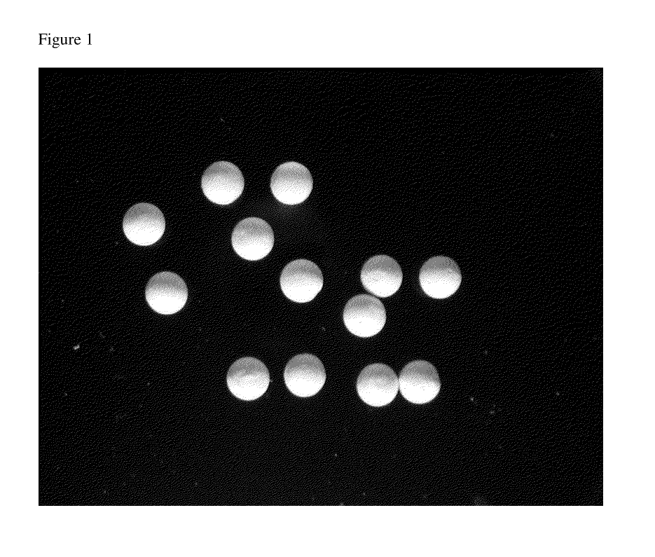

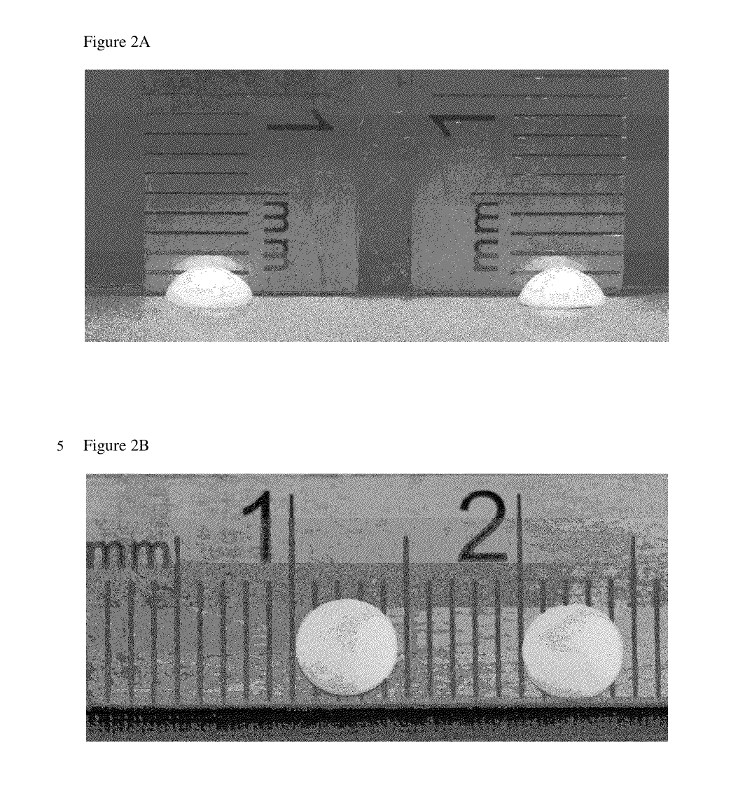

### PREFERRED EMBODIMENT OF THE INVENTION

Referring to the drawings, the solid, dry inoculum according to the invention has a substantially disc-shaped body, which comprises a convex surface opposed by a corresponding concave surface.

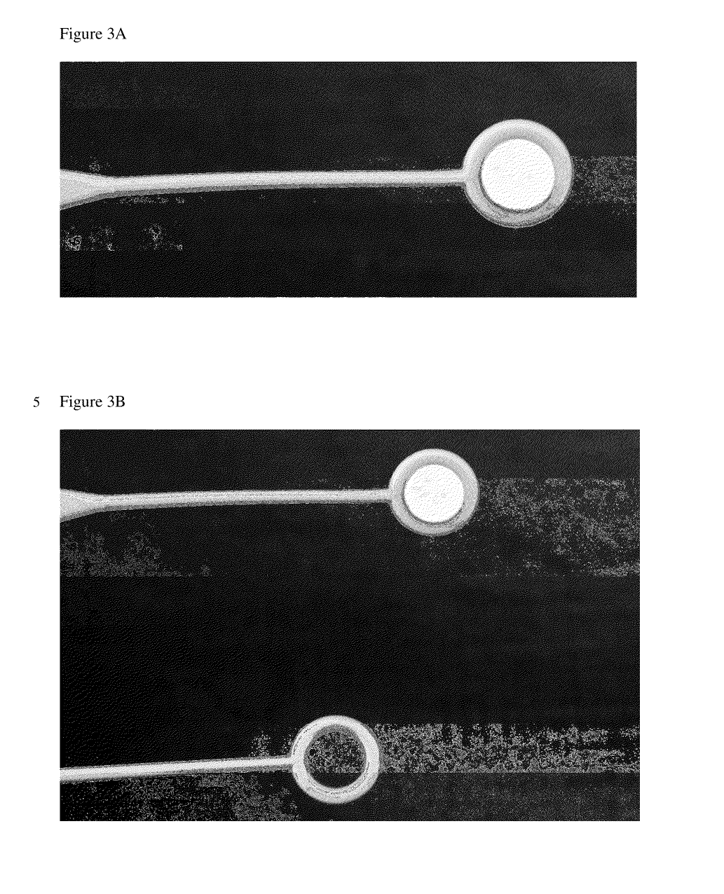

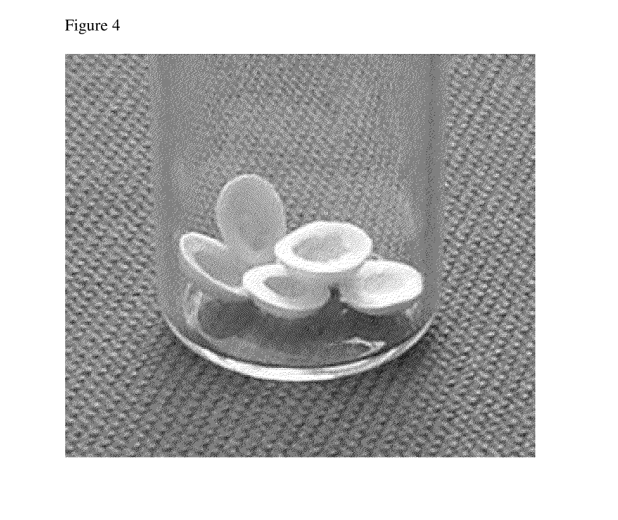

In one preferred embodiment, the particular shape of the inocula, as shown in the Figures, facilitates handling of an inoculum by a user under sterile conditions. For example, the shape allows easy handling of the inoculum using a standard microbiological inoculating loop, such as shown in FIGS. 3A and 3B, thereby facilitating the transfer of the inoculum between containers.

Further, in a preferred embodiment, the inoculum comprises a predetermined amount of inoculating material, preferably cellular microorganisms such as bacteria or fungi, in the amount of up to approximately 10⁸ cells/inoculum, and a cryopreservation medium to protect and preserve these cells in a viable state during the process of drying and subsequent storage of the inoculum. To this end, the cryopreservation medium preferably comprises Bovine Serum Albumin (BSA) and gelatin in about equal parts, but also comprises myo-inositol, beef extract, bacteriological peptone, activated charcoal and water.

In one preferred embodiment the inoculum comprises bacteria selected from the group consisting of Bacillus cereus; B. pumilus; B. subtilis; Bacteroides fragilis; Brevundimonas diminuta; Bordetella bronchiseptica; Burkholderia cepacia; Clostridium perfringens; C. sporogenes; Enterococcus faecalis; E. hirae; Escherichia coli; Geobacillus stearothermophilus; Klebsiella pneumonia; Kocuria rhizophila; Lactobacillus fermentum; Listeria monocytogenes; Micrococcus luteus; Pseudomonas aeruginosa; Salmonella enterica subsp.; Shigella flexneri; Staphylococcus aureus subsp.; and Streptococcus pyogenes.

In another preferred embodiment, the inoculum comprises fungi selected from the group consisting of Aspergillus niger; Candida albicans; and Saccharomyces cerevisiae. According to a further preferred embodiment of the invention the dried inoculum maintains a consistent degree of viability of bacterial or fungal cells over a period of several months. The degree of viability of the cells can be assessed by the number of colony forming units (CFU) produced per inoculum after a specific storage period at 4° C. Accordingly, a reduction in cell viability will be represented by a reduction in CFUs produced per inoculum (see Table 1 below).

The inoculum according to another preferred embodiment of the invention provides a quantitative and stable inoculum for the consistent and standardized inoculation of culture medium for consistently preparing fresh, equivalent reference cultures. Such reference cultures may be routinely used in microbiological quality control processes.

In another preferred embodiment, the present invention relates to a method of preparing a plurality of the substantially solid, substantially dry inocula described above. The plurality of inocula is prepared by depositing a corresponding plurality of aliquots of liquid cryopreservation medium in the form of droplets having a volume of about 10 μl to about 100 μl, preferably of about 15 μl to about 60 μl, and containing approximately 10⁸ bacterial or fungal cells, on the polystyrene surface of a standard laboratory polystyrene Petri Dish. It will be understood that the number of bacteria or fungi in the inoculum may be higher or lower, depending on the desired use of the inoculums. Each of the droplets is then frozen and subsequently freeze-dried on the polystyrene surface to obtain a solid, dry inoculum which is conveniently disc-shaped to facilitate handling with a standard microbiological inoculating loop. Specifically, after the inoculum is removed from the polystyrene surface of the Petri Dish, it includes a substantially convex surface opposed by a corresponding substantially concave surface and can easily be transferred between containers using a sterile inoculating loop as described above. The final diameter of the solid, dry inoculum will be governed to a large extent by the size of the droplets used. In one or more preferred embodiments the solid, dry inoculum when prepared by the above method can be packaged for storage and sale. While being relatively easy and inexpensive to produce, each of the inocula contained in such a package provides a stable, quantitative inoculum containing a substantially identical, predetermined amount of inoculating material.

The invention will now be described in more detail with reference to non-limiting examples.

### Examples

### 1. Cryopreservation Medium (CM)

### Components of the Cryopreservation Medium:

| | |
|---|---|
| Bovine Serum Albumin, BSA (Sigma, A9056) | 5.0% |
| myo-Inositol (Sigma, I5125) | 10.0% |
| Beef Extract (BD, 212303) | 0.3% |
| Bacteriological Peptone (Oxoid, LP0037) | 0.5% |
| Gelatin (Sigma, G9382) | 5.0% |
| Activated Charcoal (Sigma, C9157) | 0.3% |

### Preparation of Cryopreservation Medium

### CM Part A:

### Inositol Solution:

| | | |
|---|---|---|
| myo-Inositol | 10 | g |
| Beef Extract | 0.3 | g |
| Bacteriological Peptone | 0.5 | g |
| DI Water | 40 | ml |

The mixture was heated at 121° C. for 15 minutes (pH 7.4±0.2).

BSA solution:

| | | |
|---|---|---|
| Bovine Serum Albumin | 5 | g |
| DI Water | 30 | ml |

The solution was sterilized by 0.22 μm filtration.

40 ml Inositol solution and 30 ml BSA solution were mixed to produce CM Part A. 7 ml CM Part A were dispensed into sterilized plastic bottles and stored at −20° C.

### CM Part B:

| | | |
|---|---|---|
| Gelatin | 5 | g |
| Activated Charcoal | 0.3 | g |
| DI Water | 30 | ml |

The mixture was heated at 121° C. for 15 minutes.

3 ml CM Part B were dispensed into sterilized plastic bottles and stored at −20° C.

### 2. Preparation of Cells for Resuspension in Cryopreservation Medium

Method for Preparation of Staphylococcus aureus and Pseudomonas aeruginosa Cells
Staphylococcus aureus
Pseudomonas aeruginosa

These organisms were plated on Tryptone Soya Agar (TSA, CM131 Oxoid) plates and incubated for 24 hours at 37° C. for S. aureus, for 24 hours at 30° C. for P. aeruginosa to produce bacterial lawns of each microorganism.

5 ml Peptone Water (Oxoid) per plate (standard 90 mm polystyrene Petri dish) were used to wash each bacterial lawn and the suspension was transferred to a centrifuge tube and centrifuged at 4500 rpm for 6 minutes. The supernatant liquid was decanted (and discarded).

The sediment was resuspended in 10 ml peptone water and spun again; this process was repeated two times.

After the second wash the supernatant liquid was decanted, the total number of cells collected was determined by photo-spectrometry and the cells were kept for resuspension in cryopreservation medium for droplet formation as described below.

Method for Preparation of Candida albicans Cells
Candida albicans

This organism was plated on Sabouraud Dextrose Agar (SDA, CM41 Oxoid) plates and incubated for 24 hours at 30° C.

5 ml Peptone Water (Oxoid) per plate were used to wash the fungal lawn and the suspension was transferred to a centrifuge tube and centrifuged at 4500 rpm for 6 minutes. The supernatant liquid was decanted (and discarded).

The sediment was resuspended in 10 ml peptone water and spun again; this process was repeated two times.

After the second wash the supernatant liquid was decanted, the total number of cells collected was determined by photo-spectrometry and the cells were kept for resuspension in cryopreservation medium for droplet formation as described below.

Method for Preparation of Bacillus subtilis and Clostridium sporogenes Spores
Bacillus subtilis
Clostridium sporogenes

B. subtilis was plated on Tryptone Soya Agar (TSA, CM131 Oxoid) with 0.3% MnSO4 plates and incubated for 9 days at 37° C.

C. sporogenes was plated on Reinforced Clostridia Agar (RCA, CM151 Oxoid) plates and incubated under anaerobic conditions for 14 days at 37° C.

5 ml Peptone Water (Oxoid) per plate were used to wash the fungal lawn and the suspension was transferred to a centrifuge tube and centrifuged at 4500 rpm for 6 minutes. The supernatant liquid was decanted (and discarded).

The sediment was resuspended in 10 ml peptone water and spun again; this process was repeated two times.

After the second wash the supernatant liquid was decanted and, subsequently, the sediment was heated at 80° C. for 20 minutes and then cooled in ice water for 20 minutes. Spore production was checked by spore stain (P 642, Merck; Microbiology Manual 12^{th} Edition). The percentage of spores formed was confirmed to be greater than 90% by visual inspection and the spores were kept for droplet formation as described below.

Method for Preparation of Aspergillus niger Spores
Aspergillus niger

This organism was grown on Sabouraud Dextrose Agar (SDA, CM41 Oxoid) slopes (in 100 ml medical flats; Schott-Duran) and incubated at 30° C. until the entire surface of the culture becomes black.

5 ml Peptone Water (Oxoid) per flat were used to wash off the confluent growth from the flats and the suspension was transferred to a McCartney bottle (Techno Plas, Australia). The suspension was passed through paper filter (Whatman No. 2) and the filtrate was transferred to a centrifuge tube and centrifuged at 4500 rpm for 6 minutes. The supernatant liquid was then decanted (and discarded).

The sediment was resuspended in 10 ml peptone water and spun again; this process was repeated two times.

After the second wash the supernatant liquid was decanted, the total number of cells collected was determined by photo-spectrometry and the cells were kept for resuspension in cryopreservation medium for droplet formation as described below.

### 3. Droplet Formation

### Equipment and Material:

Petri Dish Polystyrene (PS), Sterilized by ethylene oxide (EO)

### Pipette: Eppendorf, Multipartite Plus Repeater Pipette

### Tip: 500 μl (25×20 μl)

### Method:

The specific amount of cell suspension was added to 7 ml CM Part A and resuspended. The cell suspension contained from about 0 to about 10⁸ bacterial or fungal cells in up to 2 ml volume collected by the methods described above (the cells initially collected were diluted in Peptone water when required).

3 ml CM Part B were added to the mixture and were mixed thoroughly by shaking. An Eppendorf Multipartite Plus pipette was set to a volume of 20 μl, and 500 μl tips were used.

20 μl bacterial CM suspension were vertically and continuously dispensed on a PS Petri Dish surface and each droplet was kept at an appropriate distance from the other to avoid accidental mixing of individual droplets.

The PS Petri Dish was then immediately moved to a −20° C. freezer for 30 minutes (pre-freezing).

After pre-freezing had been completed, the PS Petri Dish was transferred to a −80° C. freezer and left overnight.

### 4. Freeze-Drying

The freeze dryer (FD-1B-50, Bi Yi Kang, China) was prepared by lowering the condenser temperature to −50° C. for half an hour before the freeze drying cycle. The PS Petri Dish with frozen droplets was put into the freeze dryer. The cycle was set to ramp the freeze dryer shelf temperature from −10° C. to 25° C. and to apply the maximum vacuum. The cycle finished when the maximum vacuum level was reached (under 20 Pa). The lyophilized discs (disc-shaped inocula) were removed from surface by tapping the back of Petri dish. A sterile metal or plastic bacteriological loop (Techno Plas, Australia) was subsequently used to collect whole lyophilized discs into a sterilized glass bottle. The lyophilized discs were dispensed into individual sterilized vials and a freeze drying stopper (pre-sterilized) was carefully placed onto each vial ensuring that the stopper did not seal the vial. The whole vial was then placed into the freeze dryer and maximum vacuum was exerted. When the maximum vacuum level was reached (under 20 Pa), the vials were capped within the freeze dryer. Before storage, the vials were crimped with aluminum crimps.

### Freeze-Dried Discs

### Viability Study

TABLE 1

| | Before | | | | | | | | | |
|---|---|---|---|---|---|---|---|---|---|---|
| | Freeze | After Freeze Drying | | | | | | | | |
| Organisms | Drying | 0 days | 1 month | 2 months | 3 months | 4 months | 5 months | 6 months | 7 months | 8 months |
| S. aureus | 1.75E+8 ± | 1.55E+8 ± | 1.38E+8 ± | 1.39E+8 ± | 1.36E+8 ± | 1.26E+8 ± | 1.29E+8 ± | 1.22E+8 ± | 1.20E+8 ± | 1.22E+8 ± |
| | 7.3% | 10.6% | 14.8% | 12.5% | 13.7% | 12.4% | 9.9% | 12.2% | 7.5% | 9.8% |
| | 1.04E+3 ± | 9.75E+2 ± | 9.8E+2 ± | 9.62E+2 ± | 9.72E+2 ± | 9.7E+2 ± | 9.66E+2 ± | N/A | N/A | N/A |
| | 9.6% | 9.8 | 8.0% | 10.0% | 9.2% | 8.4% | 7.6% | | | |
| P. aeruginosa | 1.03E+8 ± | 3.06E+6 ± | 3.20E+6 | 3.12E+6 ± | 3.18E+6 ± | 3.08E+6 ± | 2.90E+6 ± | 2.96E+6 ± | 2.80E+6 ± | 2.86E+6 ± |
| | 10.3% | 14.2% | 14.4% | 15.0% | 14.6% | 14.6% | 14.9% | 9.3% | 12.6.0% | 14.5% |
| | 2.68E+4 ± | 8.62E+2 ± | 8.32E+2 ± | 7.93E+2 ± | 8.02E+2 ± | 8.15E+2 ± | 8.08E+2 ± | N/A | N/A | N/A |
| | 9.4% | 13.6% | 9.8% | 8.9% | 11.2% | 9.0% | 8.9% | | | |
| C. albicans | 1.16E+7 ± | 4.16E+6 ± | 4.17E+6 ± | 3.66E+6 ± | 3.92E+6 ± | 3.51E+6 ± | 3.62E+6 ± | N/A | N/A | N/A |
| | 14.0% | 14.2% | 16.2% | 13.2% | 14.8% | 13.8% | 14.2% | | | |
| | 2.54E+3 ± | 8.64E+2 ± | 7.82E+2 ± | 8.20E+2 ± | 7.62E+2 ± | 7.20E+2 ± | N/A | N/A | N/A | N/A |
| | 12.5% | 9.9% | 9.7% | 9.0% | 9.6% | 10.3% | | | | |
| E. coli | 1.79E+8 ± | 1.29E+8 ± | 1.27E+8 ± | 1.30E+8 ± | 1.24E+8 ± | 1.20E+8 ± | 1.18E+8 ± | 1.22E+8 ± | 1.21E+ ± | 1.23E+8 ± |
| | 7.3% | 14.5% | 13.7% | 12.0% | 10.2% | 13.0% | 14.3% | 13.2% | 13.2% | 15.0 |
| | 1.20E+3 ± | 8.69E+2 ± | 8.81E+2 ± | 7.51E+2 ± | 7.86E+2 ± | 7.81E+2 ± | 7.60E+2 ± | N/A | N/A | N/A |
| | 12.6% | 11.5% | 14.2% | 13.0% | 14.7% | 13.3% | 12.8% | | | |
| B. subtilis | 1.50E+7 ± | 1.61E+7 ± | 1.60E+7 ± | 1.58E+7 ± | 1.66E+7 ± | 1.60E+7 ± | 1.52E+ ± | 1.56E+7 ± | N/A | N/A |
| | 12.2% | 9.9% | 9.3% | 9.6% | 7.8% | 10.1% | 9.4% | 10.8% | | |
| | 1.04E+3 ± | 8.65E+2 ± | 8.65E+2 ± | 8.05E+2 ± | 8.40E+2 ± | 8.25E+2 ± | N/A | N/A | N/A | N/A |
| | 10.5% | 9.5% | 9.4% | 10.4% | 9.6& | 9.0% | | | | |
| C. sporogenes | 9.82E+2 ± | 8.63E+2 ± | 9.02E+2 ± | 8.88E+2 ± | 8.82E+2 ± | 8.68E+2 ± | N/A | N/A | N/A | N/A |
| | 11.2% | 11.9% | 7.0% | 9.1% | 8.9% | 8.2% | | | | |
| A. niger | 4.62E+2 ± | 4.08E+2 ± | 4.02E+2 ± | 4.00E+2 ± | 4.32E+2 ± | 4.20E+2 ± | N/A | N/A | N/A | N/A |
| | 7.6% | 7.8% | 7.5% | 8.2% | 7.7% | 7.3% | | | | |

Vials were stored at 4° C.
Values are expressed as colony forming units per disc (CFU/disc)
N/A = not assessed;
CFU counting method: Pour Plate.

It will be appreciated that the illustrated invention provides a substantially solid, dry inoculum which is inexpensive and is sized and shaped for more convenient use. Although the invention has been described with reference to specific examples, it will be appreciated by those skilled in the art that the invention may be embodied in many other forms.

1. A solid, freeze-dried, standardized inoculum comprising a cryopreservation medium and a predetermined amount of a microorganism,
wherein said inoculum is substantially disc-shaped comprising a convex surface, wherein said inoculum is sized and shaped so as to facilitate its transfer between containers using a microbiological inoculating loop, and
wherein said inoculum is suitable for producing a reference culture of said microorganism.

2. The inoculum according to claim 1, wherein said convex surface is opposed by a corresponding concave surface.

3. (canceled)

4. The inoculum according to claim 1, wherein said cryopreservation medium comprises Bovine Serum Albumin and gelatin in about equal parts.

5. The inoculum according to claim 1, wherein said cryopreservation medium comprises Bovine Serum Albumin, myo-inositol, beef extract, bacteriological peptone, gelatin, activated charcoal and water.

6. (canceled)

7. The inoculum according to claim 1, wherein said inoculum comprises about 10³ microorganisms.

8. The inoculum according to claim 1, wherein said inoculum comprises about 10² microorganisms.

9. (canceled)

10. The inoculum according to claim 1, wherein said microorganism is selected from the group consisting of bacteria and fungi.

11. The inoculum according to claim 10, wherein said bacteria are selected from the group consisting of Bacillus cereus; B. pumilus; B. subtilis; Bacteroides fragilis; Brevundimonas diminuta; Bordetella bronchiseptica; Burkholderia cepacia; Clostridium perfringens; C. sporogenes; Enterococcus faecalis; E. hirae; Escherichia coli; Geobacillus stearothermophilus; Klebsiella pneumonia; Kocuria rhizophila; Lactobacillus fermentum; Listeria monocytogenes; Micrococcus luteus; Pseudomonas aeruginosa; Salmonella enterica subsp.; Shigella flexneri; Staphylococcus aureus subsp.; and Streptococcus pyogenes.

12. The inoculum according to claim 10, wherein said fungi are selected from the group consisting of Aspergillus niger; Candida albicans; and Saccharomyces cerevisiae.

13. A method of preparing a solid, freeze-dried, standardized inoculum comprising the steps of:
a) depositing a plurality of aliquots of liquid cryopreservation medium comprising a predetermined amount of a microorganism on a surface;
b) freeze-drying each of said aliquots on said surface to obtain a plurality of solid, dry inocula; and
c) removing said inocula from said surface,
wherein each said inoculum is substantially disc-shaped comprising a convex surface,
wherein said inoculum is sized and shaped so as to facilitate its transfer between containers using a microbiological inoculating loop, and
wherein said inoculum is suitable for producing a reference culture of said microorganism.

14. The method according to claim 13, wherein said convex surface is opposed by a corresponding concave surface.

15. (canceled)

16. The method according to claim 13, wherein each of said aliquots has a volume of about 10 μl to about 100 μl.

17. The method according to claim 16, wherein each of said aliquots has a volume of about 15 μl to about 60 μl.

18. The method according to claim 13, wherein said surface is a plastic surface.

19. The method according to claim 18, wherein said surface is a polystyrene surface.

20. (canceled)

21. The method according to claim 13, wherein said inoculum comprises about 10³ microorganisms.

22. The method according to claim 13, wherein said inoculum comprises about 10² microorganisms.

23. (canceled)

24. The method according to claim 13, wherein said microorganism is selected from the group consisting of bacteria and fungi, and
wherein, optionally,
said bacteria are selected from the group consisting of Bacillus cereus; B. pumilus; B. subtilis; Bacteroides fragilis; Brevundimonas diminuta; Bordetella bronchiseptica; Burkholderia cepacia; Clostridium perfringens; C. sporogenes; Enterococcus faecalis; E. hirae; Escherichia coli; Geobacillus stearothermophilus; Klebsiella pneumonia; Kocuria rhizophila; Lactobacillus fermentum; Listeria monocytogenes; Micrococcus luteus; Pseudomonas aeruginosa; Salmonella enterica subsp.; Shigella flexneri; Staphylococcus aureus subsp.; and Streptococcus pyogenes, and
wherein, optionally,
said fungi are selected from the group consisting of Aspergillus niger; Candida albicans; and Saccharomyces cerevisiae.

25. (canceled)

26. (canceled)

27. A substantially solid, freeze-dried, standardized inoculum when prepared by the method according to claim 13.

28. A package containing a substantially solid, substantially dry inoculum according to claim 1.

29. (canceled)
